(19) United States
(12) Patent Application Publication
(10) Pub. No.: US 2025/0282716 A1
(43) Pub. Date: Sep. 11, 2025

(54) METHODS OF MAKING IP-RECEPTOR AGONISTS

(71) Applicant: United Therapeutics Corporation, Silver Spring, MD (US)

(72) Inventors: Hitesh Batra, Herndon, VA (US); Sudersan Tuladhar, Silver Spring, MD (US); Sri Harsha Tummala, Owings Mills, MD (US); Mark Farquharson, Vienna, VA (US)

(73) Assignee: United Therapeutics Corporation, Silver Spring, MD (US)

(21) Appl. No.: 19/074,841

(22) Filed: Mar. 10, 2025

Related U.S. Application Data
(60) Provisional application No. 63/563,664, filed on Mar. 11, 2024.

(51) Int. Cl. C07C 269/06; C07C 271/28

(52) U.S. Cl. CPC C07C 269/06; C07C 271/28

(57) ABSTRACT

Alternative processes of synthesizing ralinepag and its salts, as well as intermediates used in such processes, are described.

### RELATED APPLICATIONS

The present application claims priority to U.S. provisional application No. 63/563,664 filed Mar. 11, 2024, which is incorporated herein by reference in its entirety.

### FIELD

The present disclosure relates methods of synthesizing prostacyclin (PGI2 or IP) receptor agonists and more specifically, to methods of synthesizing ralinepag and its salts as well to intermediates for synthesizing ralinepag and its salts.

### SUMMARY

An embodiment is a method of synthesizing a compound of formula (1):

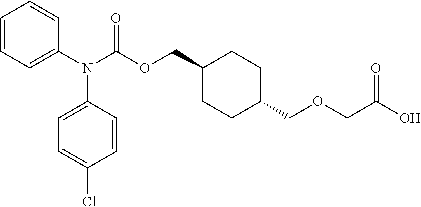 [SMILES: O=C(O)COC[C@H]1CC[C@H](COC(=O)N(c2ccccc2)c2ccc(Cl)cc2)CC1]

or a salt thereof, the method comprising:
(a) converting a carbamate compound of formula (5):

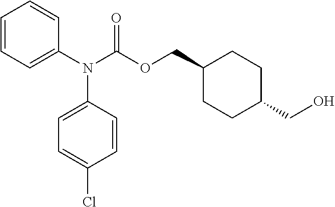 [SMILES: O=C(OC[C@H]1CC[C@H](CO)CC1)N(c1ccccc1)c1ccc(Cl)cc1]

into a triflate compound of formula (8):

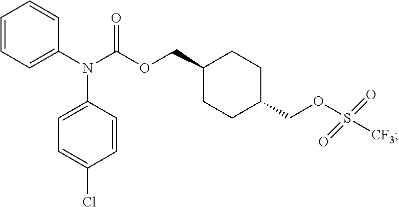 [SMILES: O=C(OC[C@H]1CC[C@H](COS(=O)(=O)C(F)(F)F)CC1)N(c1ccccc1)c1ccc(Cl)cc1]

(b) converting the triflate compound of formula (8) into a methyl ester compound of formula (9):

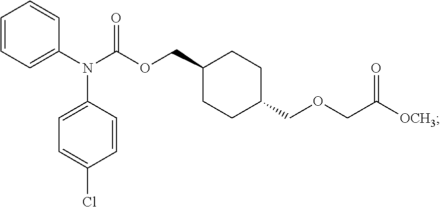 [SMILES: COC(=O)COC[C@H]1CC[C@H](COC(=O)N(c2ccccc2)c2ccc(Cl)cc2)CC1]

and
(c) converting the methyl ester compound of formula (9) into a salt compound of formula (7):

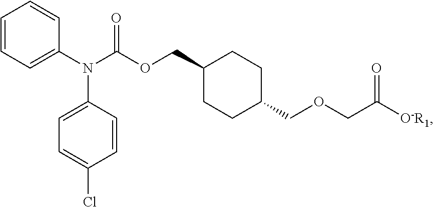

wherein R₁ is a cation selected from Na⁺, K⁺, and NH₄⁺.

Another embodiment is a method of synthesizing a compound of formula (1):

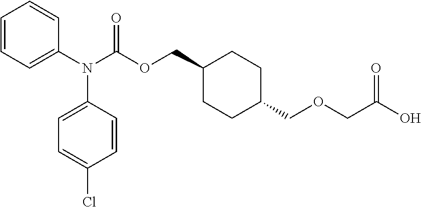 [SMILES: O=C(O)COC[C@H]1CC[C@H](COC(=O)N(c2ccccc2)c2ccc(Cl)cc2)CC1]

or a salt thereof, the method comprising:
(a) reacting a compound of formula (3):

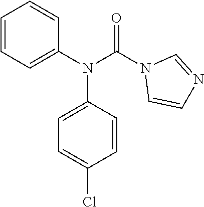 [SMILES: O=C(N(c1ccccc1)c1ccc(Cl)cc1)n1ccnc1]

with a compound of formula (10)

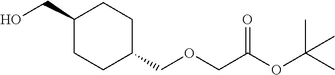 [SMILES: CC(C)(C)OC(=O)COC[C@H]1CC[C@H](CO)CC1]

to form a compound of formula (6):

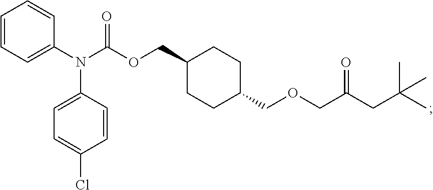 [SMILES: CC(C)(C)CC(=O)COC[C@H]1CC[C@H](COC(=O)N(c2ccccc2)c2ccc(Cl)cc2)CC1]

(b) converting the compound of formula (6) into a compound of formula (11):

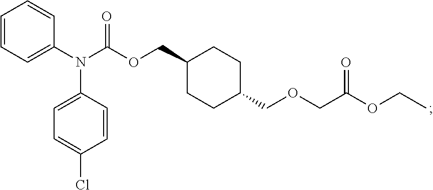 [SMILES: CCOC(=O)COC[C@H]1CC[C@H](COC(=O)N(c2ccccc2)c2ccc(Cl)cc2)CC1]

and
(c) converting the compound of formula (11) into a salt compound of formula (7):

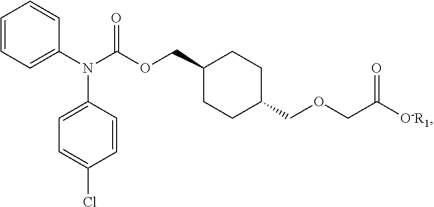

wherein R₁ is a cation selected from Na⁺, K⁺, and NH₄⁺.

Another embodiment is a method of synthesizing a compound of formula (1):

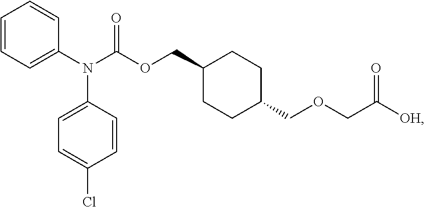 [SMILES: O=C(O)COC[C@H]1CC[C@H](COC(=O)N(c2ccccc2)c2ccc(Cl)cc2)CC1]

the method comprises:
(a) reacting a compound of formula (3):

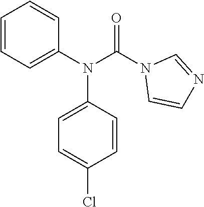 [SMILES: O=C(N(c1ccccc1)c1ccc(Cl)cc1)n1ccnc1]

with a compound of formula (10)

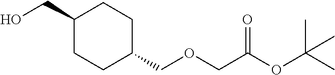 [SMILES: CC(C)(C)OC(=O)COC[C@H]1CC[C@H](CO)CC1]

to form a compound of formula (6):

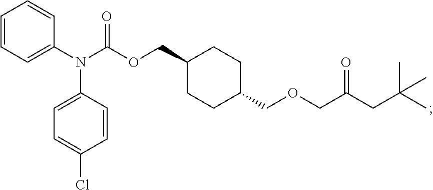 [SMILES: CC(C)(C)CC(=O)COC[C@H]1CC[C@H](COC(=O)N(c2ccccc2)c2ccc(Cl)cc2)CC1]

and
(b) reacting the compound of formula (6) with a base to form a salt compound of formula (7):

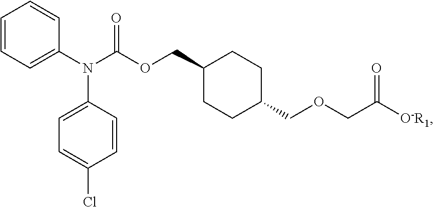

wherein R₁ is a cation selected from Na⁺, K⁺, and NH₄⁺.

Another embodiment is a compound of formula (10):

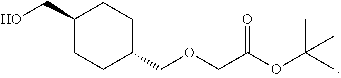 [SMILES: CC(C)(C)OC(=O)COC[C@H]1CC[C@H](CO)CC1]

Another embodiment is a compound of formula (11):

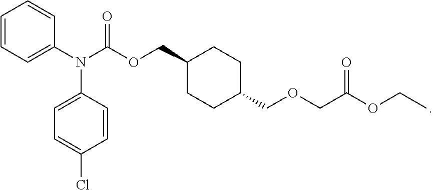 [SMILES: CCOC(=O)COC[C@H]1CC[C@H](COC(=O)N(c2ccccc2)c2ccc(Cl)cc2)CC1]

Another embodiment is a batch of a compound of formula (6):

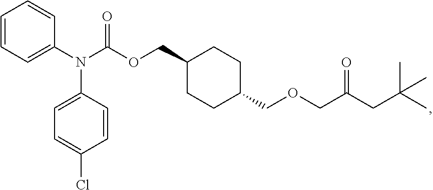 [SMILES: CC(C)(C)CC(=O)COC[C@H]1CC[C@H](COC(=O)N(c2ccccc2)c2ccc(Cl)cc2)CC1]

wherein said batch contains less than 1% of a dimer impurity of the following formula:

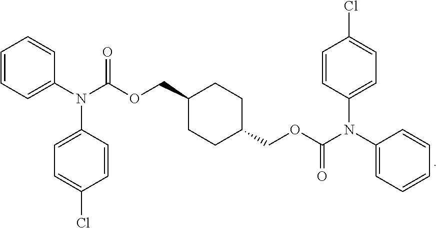 [SMILES: O=C(OC[C@H]1CC[C@H](COC(=O)N(c2ccccc2)c2ccc(Cl)cc2)CC1)N(c1ccccc1)c1ccc(Cl)cc1]

Another embodiment is a batch of a compound of formula (1):

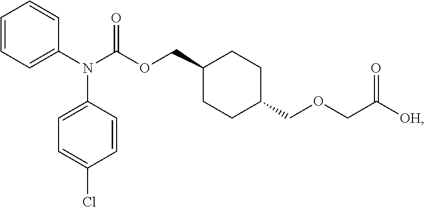 [SMILES: O=C(O)COC[C@H]1CC[C@H](COC(=O)N(c2ccccc2)c2ccc(Cl)cc2)CC1]

wherein said batch has purity of at least 99.9%.

Yet another embodiment is a method of producing a compound of formula (1):

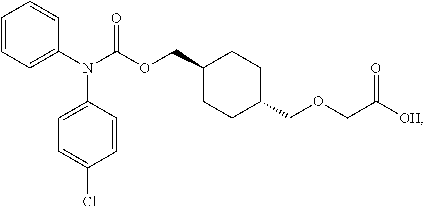 [SMILES: O=C(O)COC[C@H]1CC[C@H](COC(=O)N(c2ccccc2)c2ccc(Cl)cc2)CC1]

the method comprising:
(a) reacting a crude product comprising a salt compound of formula (7):

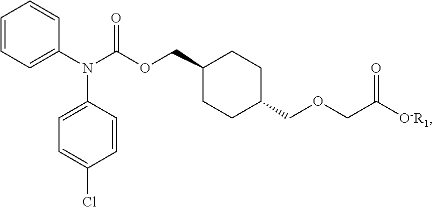

wherein R₁ is a cation selected from Na⁺, K⁺, and NH₄⁺ with an acid to form a crude product comprising the compound of formula (1); and
(b) crystallizing the compound of formula (1) from the crude product formed in the reacting.

### FIGURES

FIG. 1 shows an alternative process for synthesizing ralinepag (Comparative Scheme 1).

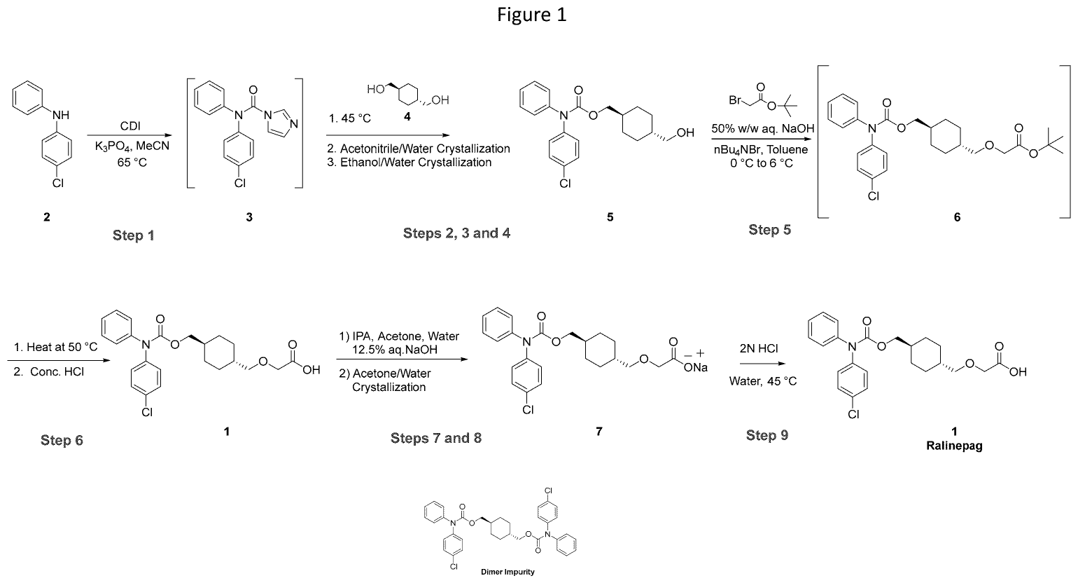

FIG. 2 shows a process for synthesizing ralinepag according to one embodiment of the present application (Scheme 2).

FIG. 3 shows a process for synthesizing ralinepag according to another embodiment of the present application (Scheme 3).

FIG. 4 shows a process for synthesizing ralinepag according to yet another embodiment of the present application (Scheme 4).

Experimental conditions in FIG. 1-4 are exemplary and non-limiting. Modifications to and variations of the exemplary conditions would be apparent to a skilled artisan.

FIG. 5 provides a comparison between Schemes 1-4, with purities measured as HPLC % area.

FIG. 6 shows a modification of the process of Scheme 4, in which the final two steps of salt crystallization and neutralization are replaced with crude salt neutralization and subsequent crude ralinepag crystallization. A similar replacement may be performed for the final two steps in each of Schemes 1-4.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims.

In order that the present disclosure can be more readily understood, certain terms are first defined. Additional definitions are set forth throughout the detailed description.

All numerical designations, e.g., pH, temperature, time, concentration, and molecular weight, including ranges, are approximations which are varied (+) or (−) by increments of 0.05%, 1%, 2%, 5%, 10% or 20%. It is to be understood, although not always explicitly stated that all numerical designations are preceded by the term “about.” It also is to be understood, although not always explicitly stated, that the reagents described herein are merely exemplary and that equivalents of such are known in the art.

“Pharmaceutically acceptable salt” refers to salts of a compound, which salts are suitable for pharmaceutical use and are derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “Handbook of Pharmaceutically Acceptable Salts,” (2002), Verlag Helvetica Chimica Acta, Zurich, Switzerland), for a discussion of pharmaceutical salts, their selection, preparation, and use.

“Pulmonary hypertension” refers to all forms of pulmonary hypertension, WHO Groups 1-5. Pulmonary arterial hypertension, also referred to as PAH, refers to WHO Group 1 pulmonary hypertension. PAH includes idiopathic, heritable, drug- or toxin-induced, and persistent pulmonary hypertension of the newborn (PPHN). Pulmonary hypertension, as used herein, also refers to the specific types of pulmonary hypertension in WHO Groups 1-5, including subtypes. For example, a reference to “pulmonary hypertension” refers to individually to PAH (WHO Group 1) and pulmonary hypertension associated with interstitial lung disease (PH-ILD) (WHO Group 3).

Generally, pharmaceutically acceptable salts are those salts that retain substantially one or more of the desired pharmacological activities of the parent compound and which are suitable for in vivo administration. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Pharmaceutically acceptable salts include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

Ralinepag refers to “2-([(1r,4r)-4-(((4-chlorophenyl)(phenyl)carbamoyloxy)methyl)-cyclohexyl]methoxy)acetic acid”, “Acetic acid, 2-((trans-4-(((((4-chlorophenyl)phenylamino)-carbonyl)oxy)methyl)cyclohexyl)methoxy)-”, “2-((trans-4-((((4-Chlorophenyl)(phenyl)carbamoyl)-oxy)methyl)cyclohexyl)methoxy)acetic acid”, or “APD-811”.

In this application, ralinepag is referred to as a compound of formula (1):

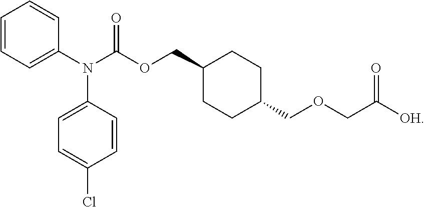 [SMILES: O=C(O)COC[C@H]1CC[C@H](COC(=O)N(c2ccccc2)c2ccc(Cl)cc2)CC1]

Ralinepag, its method of synthesizing and its various uses are disclosed in PCT application publications Nos. WO2009/117,095 and WO2011/037613; U.S. Pat. Nos. 10,537,546, 11,123,298, and 11,826,471; U.S. Patent application publications Nos. US20110053598, US20200368190, US20190321328, US20180303789, US20240002334, and US20230406815, each of which is incorporated herein by reference in its entirety, including for their descriptions of synthesizing ralinepag and its precursors and variants.

Comparative Scheme 1 illustrates an alternative process for synthesizing ralinepag.

The process of Comparative Scheme 1 includes nine steps:

Step 1: reacting an aniline compound of formula (2):

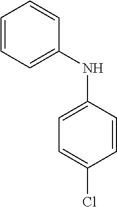 [SMILES: Clc1ccc(Nc2ccccc2)cc1]

with carbonyldiimidazole to form a compound of formula (3):

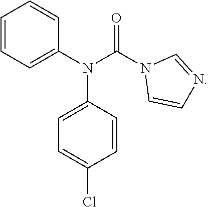 [SMILES: O=C(N(c1ccccc1)c1ccc(Cl)cc1)n1ccnc1]

This reaction may be performed in the presence of a coupling agent, such as 1,1′-Carbonyldiimidazole (CDI). This reaction may be performed in the presence of a base, such as potassium phosphate (K₃PO₄). A solvent for this reaction may be, for example, acetonitrile (MeCN), though other solvents could be employed such as tetrahydrofuran, 2-methyl tetrahydrofuran, toluene, dichloromethane. The reaction may be performed at a temperature of about 65° C.

Step 2: reacting the compound of formula (3) with a compound of formula (4):

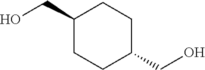

to form a product comprising a carbamate compound of formula (5):

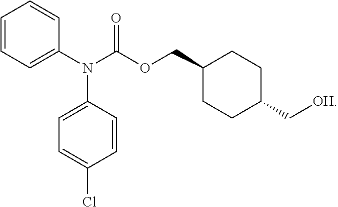 [SMILES: O=C(OC[C@H]1CC[C@H](CO)CC1)N(c1ccccc1)c1ccc(Cl)cc1]

This reaction may be performed at a temperature of about 45° C. A solvent for this reaction may be the same as the solvent in Step 1.

Steps 3 and 4 may be two crystallizations of the product of the reaction of step 2 to increase a purity of the compound of formula (5). For example, the two crystallizations may be used to produce the compound of formula (5) in a solid form. The two crystallizations may be performed using different solvents or mixtures of solvents. For example, the first crystallization of step 3 may use a first solvent or a first mixture of solvents, while the second crystallization of step 4 may use a second solvent or a second mixture of solvents. In some embodiments, the first crystallization of Step 3 may use a first organic solvent, which may be an aprotic polar solvent, such as acetonitrile, alone or in mixture with water, while the second crystallization of Step 4 may use a second organic solvent, which may be a protic polar solvent, such as low alcohol, e.g. methanol, ethanol, propanol or isopropanol, alone or in mixture in water.

Step 5 of Scheme 1 involves reacting the crystallized compound of formula (5) with

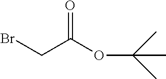

to form a product including the compound of formula (6). This reaction has to be performed in a strong base such as about 50% w/w aqueous NaOH or KOH. This reaction can be performed in the presence of tetrabutylammonium bromide (TBAB, nBu₄NBr), a phase transfer catalyst. A solvent for this reaction can be toluene, methyl tert-butyl ether, or dichloromethane. A temperature for this reaction can be from about 0° C. to about 6° C.

Step 6 of Scheme 1 involves hydrolyzing, with a first hydrolysis of the product of Step 5 that includes the compound of formula (6) at 50° C. to form a crude product that includes ralinepag, i.e., a compound of formula (1). The neutralization reaction uses a concentrated acid, such as concentrated 12N HCl. Neutralization is performed at 0° C. to RT.

Step 7 involves converting the crude product of Step 6 into a crude product that contains ralinepag salt, i.e. the compound of formula 7. The conversion may be performed using a diluted base, i.e. base diluted in water, such as diluted KOH or diluted NaOH. A concentration of the base may be from about 10% to about 15%, such as about 12.5% (mass %). The conversion may be performed in a solvent, which may include one or more of acetone, isopropanol (IPA) and water. In many embodiments, the solvent may include acetone, isopropanol and water.

Step 8 involves crystallizing the crude product of Step 7 that contains the ralinepag salt, i.e. the compound of formula 7, to produce a crystalized product with a higher purity of the ralinepag salt. The crystallization may be performed in an aprotic solvent, such as acetone, or in a mixture of an aprotic solvent, such as acetone, with water.

Step 9 involves converting the crystallized product of step 7 that contains the ralinepag salt back into a product containing ralinepag. The ralinepag containing product of Step 9 has a much higher purity of ralinepag than the crude product of Step 6. The conversion may be performed using a diluted acid, such as diluted HCl, e.g. about 2N HCl. A temperature of the conversion may be about 45° C.

The process of Comparative Scheme 1 has the following drawbacks:
(1) Dimer Impurity: A dimer impurity is formed in Step 2 of Scheme 1 in Step 2 due to two free hydroxy groups on the diol compound of formula (4). The dimer is a critical impurity which is usually removed by a separate step by ethanol/water crystallization, see step 3 in Scheme 1. Also, the removal of the dimer impurity may be challenging and may involve multiple in-process testing to monitor its removal. In the process of Scheme 1, the dimer impurity may range from 1-3% in the isolated crude carbamate (5) before acetonitrile/water crystallization and ethanol/water crystallization.
(2) Carbamate Impurity: A carbamate impurity of formula (5) impurity is formed in 7-8% range during the ralinepag t-butyl ester (6) hydrolysis in Step 6 of Scheme 1. This is formed due to large excess of sodium hydroxide. This decreases the yield due to loss of product, and this impurity is a burden to remove at the ralinepag sodium salt crystallization step using acetone/water.
(3) Use of Harsh Conditions: Harsh conditions are used in the current process, such as 50% NaOH (40-44 eq.) in water for alkylation of carbamate (5) in Step 5 and concentrated HCl for neutralization of ralinepag sodium salt formed after the hydrolysis of ralinepag t-butyl ester (6) in Step 6. The use of these harsh conditions could pose challenges, such as high exotherm and operator safety during scale-up and scalability of the process due to excess volumes.
(4) Redundant Step: The hydrolysis of ralinepag t-butyl ester (6) Step 6 generates ralinepag sodium salt, however in the process of Scheme 1, it cannot be isolated due to presence of large amounts of sodium hydroxide and the pasty nature of the ralinepag sodium salt in toluene/water mixture. Thus, the crude ralinepag sodium salt is neutralized to ralinepag acid using concentrated HCl and then again converted back to crude ralinepag sodium salt in a separate step (Step 7). This is a redundant step in the current process of Scheme 1.
(5) Number of Steps: Due to the above challenges multiple crystallizations, isolations and drying steps are needed in this process to obtain desired quality of final ralinepag. These steps also contribute to increased campaign time and in-process testing creating in efficiencies.
(6) Yield: The overall yield of the current process in Scheme 1 is also relatively low (approx. 45%).

The present disclosure provides alternative processes for synthesizing ralinepag, which address one or more of drawbacks of the process of Comparative Scheme 1.

According to one embodiment, an alternative process for synthesizing ralinepag may involve:
(a) converting a carbamate compound of formula (5):

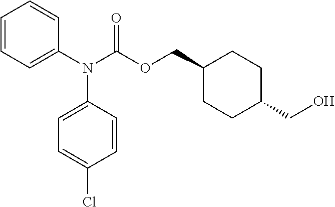 [SMILES: O=C(OC[C@H]1CC[C@H](CO)CC1)N(c1ccccc1)c1ccc(Cl)cc1]

into a triflate compound of formula (8):

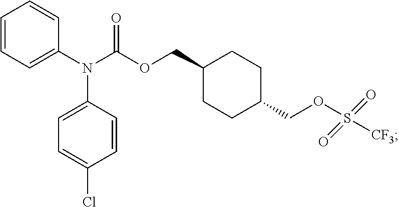 [SMILES: O=C(OC[C@H]1CC[C@H](COS(=O)(=O)C(F)(F)F)CC1)N(c1ccccc1)c1ccc(Cl)cc1]

(b) converting the triflate compound of formula (8) into a methyl ester compound of formula (9):

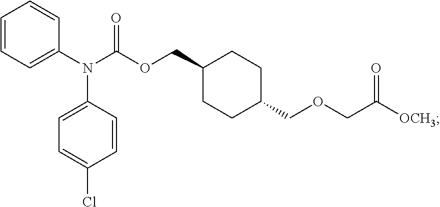 [SMILES: COC(=O)COC[C@H]1CC[C@H](COC(=O)N(c2ccccc2)c2ccc(Cl)cc2)CC1]

and
(c) converting the methyl ester compound of formula (9) into a salt compound of formula (7):

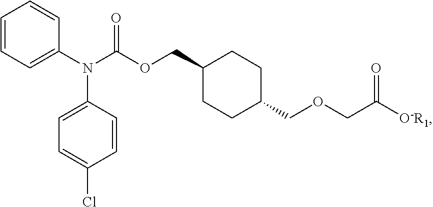

where R₁ may a cation, such as Na⁺, K⁺, or NH₄⁺, though any base that is capable of hydrolyzing an ester functional group can be used, such as sodium hydroxide, potassium hydroxide, cesium hydroxide, lithium hydroxide, calcium hydroxide, or magnesium hydroxide.

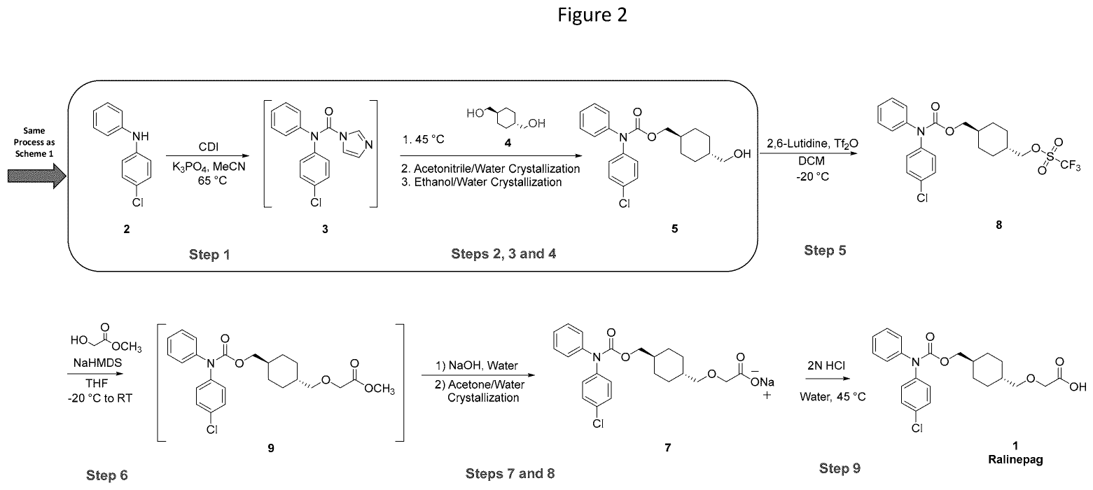

One embodiment of an alternative process is illustrated in Scheme 2 in FIG. 2. Scheme 2 has 9 steps.

Steps 1-4 in Scheme 2 may be the same or similar as Step 1-4 in Scheme 1.

Step 5 of Scheme 2 may involve converting the carbamate compound of formula (5):

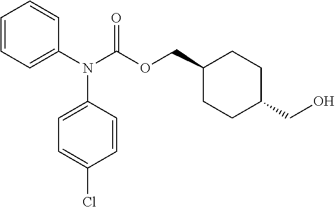 [SMILES: O=C(OC[C@H]1CC[C@H](CO)CC1)N(c1ccccc1)c1ccc(Cl)cc1]

into a crude product containing the triflate compound of formula (8):

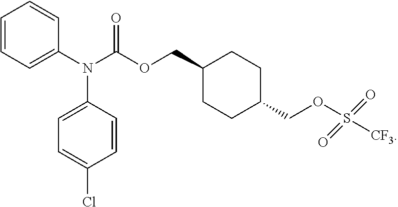 [SMILES: O=C(OC[C@H]1CC[C@H](COS(=O)(=O)C(F)(F)F)CC1)N(c1ccccc1)c1ccc(Cl)cc1]

Such conversion may be performed by reacting the compound of formula 5 with trifluoromethanesulfonic anhydride ((CF₃SO₂)₂O or Tf₂O). Such reaction may be performed in the presence of a non-nucleophilic base or inorganic base, such as 2,6-lutidine, though other bases such as potassium carbonate can be used. The reaction may be performed in a solvent, such as dichloromethane (DCM). Other solvents can be used such as dichloroethane, chloroform, or dimethylformamide. A temperature for this reaction may be, for example, about −20° C.

Step 6 of Scheme 2 may involve converting the crude product containing the triflate compound of formula (8) into the methyl ester compound of formula (9)

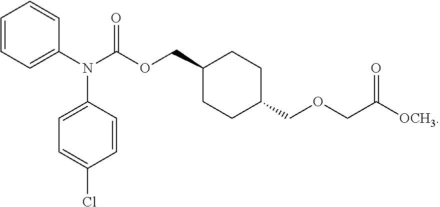 [SMILES: COC(=O)COC[C@H]1CC[C@H](COC(=O)N(c2ccccc2)c2ccc(Cl)cc2)CC1]

Such conversion may involve reacting the triflate compound of formula (8) with methyl glycolate:

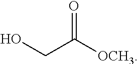

Such reaction may be performed in the presence of a base, such as sodium bis(trimethylsilyl)amide (NaHMDS). A solvent for such reaction may be tetrahydrofuran (THF). Other solvents that may be used include methyl tert-butyl ether, 2-methyl tetrahydrofuran, or dimethylformamide. The reaction may start at a temperature of about −20° C., while allowing warming up to a room temperature, such as about 20° C. or about 25° C.

Step 7 in Scheme may involve converting the methyl ester compound of formula (9) in a product comprising the salt compound of formula (7):

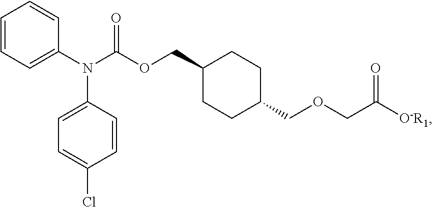

where R₁ may be a cation such as Na⁺, K⁺, or NH₄⁺. Such conversion may involve reacting the ethyl ester compound of formula (9) with a base, such as NaOH, KOH or NH₄OH.

Step 8 in Scheme 2 may involve crystallizing the salt compound of formula (7) from the product of the reaction of Step 7 to improve the purity of the salt compound of formula 7 and/or to obtain the salt compound of formula 7 in a crystalline form. Such crystallization may be performed from an organic solvent, such as an aprotic organic solvent, e.g. acetone, alone or in mixture with water.

Step 9 of Scheme 2 may be the same or similar as Step 9 of Scheme 1.

Highlights and/or advantages of the process of Scheme 2 may be as follows:
(1) The first few steps (Steps 1-4) may be the same as in the process of Scheme 1 up to the isolation of carbamate compound of formula (5).
(2) In the process of Scheme 2 process, the carbamate compound of formula (5) is converted to a carbamate triflate compound of formula (8) in Step 5, which is carried crude to the next step (Step 6) after the isolation.
(3) The crude triflate (8) is converted to ralinepag ethyl ester (9) in Step 6. The ethyl ester group is relatively easy to hydrolyze without the use of harsh conditions unlike ralinepag t-butyl ester (6) intermediate of Scheme 1 process. No large excess of 50% aq. sodium hydroxide is used to hydrolyze and hence no concentrated HCl is needed for neutralization.
(4) The formation of carbamate impurity is alleviated (7-8% in Scheme 1 process vs 0.88% in Scheme 2 process) in the hydrolysis step as relatively very low equivalents (42 eq. in Scheme 1 process vs 2.75 eq. in Scheme 2 process) of sodium hydroxide is sufficient. This reduced the burden on the subsequent acetone/water crystallization step.
(5) Since low equivalents of sodium hydroxide was used, the crude ralinepag sodium salt was isolated from the reaction mixture in Step 7. The redundant sodium salt reformation step is avoided as the crude ralinepag sodium salt was directly isolated from the reaction mixture unlike Scheme 1 process.
(6) The harsh conditions were avoided as only 2.75 eq. of sodium hydroxide was used for the hydrolysis of ralinepag ethyl ester (9) improving the safety of the process, ease of isolation of sodium salt and scalability of the process.

In some embodiments, an alternative process for synthesizing ralinepag may involve:
(a) reacting a compound of formula (3):

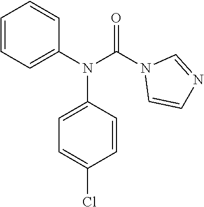 [SMILES: O=C(N(c1ccccc1)c1ccc(Cl)cc1)n1ccnc1]

with a compound of formula (10)

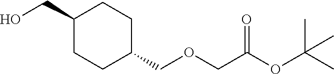 [SMILES: CC(C)(C)OC(=O)COC[C@H]1CC[C@H](CO)CC1]

to form a compound of formula (6):

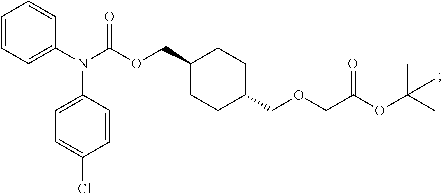 [SMILES: CC(C)(C)OC(=O)COC[C@H]1CC[C@H](COC(=O)N(c2ccccc2)c2ccc(Cl)cc2)CC1]

(b) converting the compound of formula (6) into a compound of formula (11):

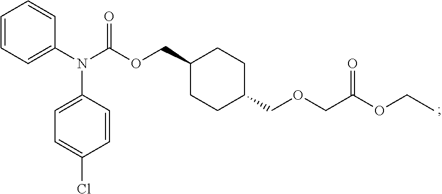 [SMILES: CCOC(=O)COC[C@H]1CC[C@H](COC(=O)N(c2ccccc2)c2ccc(Cl)cc2)CC1]

and
(c) converting the compound of formula (11) into a salt compound of formula (7):

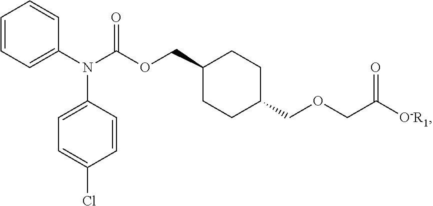

where R₁ may be a cation, such as Na⁺, K⁺, or NH₄⁺.

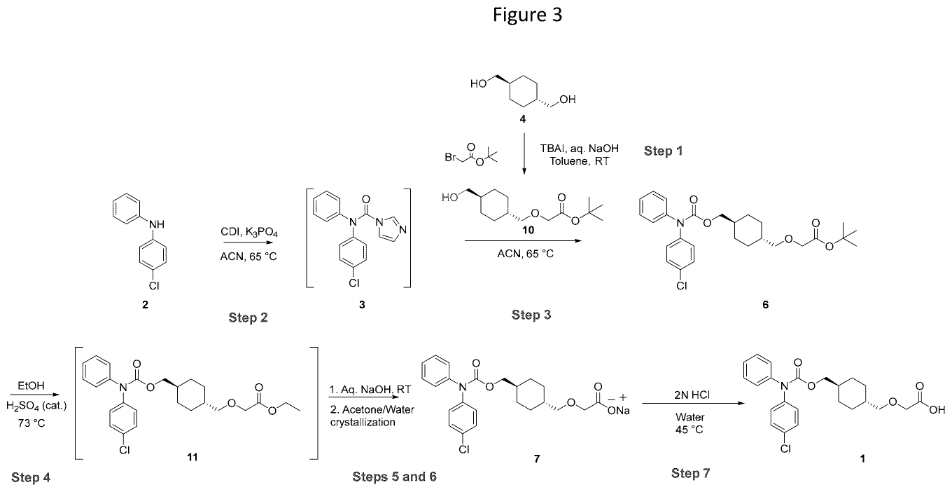

One embodiment of such alternative process is illustrated in Scheme 3 on FIG. 3.

Step 1 of Scheme 3 may involve reacting a compound of formula (4):

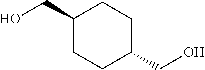

with tert-butyl bromoacetate

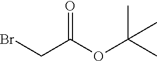

to form the compound of formula (10). Such reaction may be performed in the presence of a phase transfer catalyst, such as tetra-n-butylammonium iodide (TBAI) and/or an aqueous solution of a base, such as NaOH or KOH. The reaction may be performed in a solvent, such as an organic solvent, e.g., toluene, methyl tert-butyl ether, dichloromethane, 2-methyl tetrahydrofuran, or tetrahydrofuran. The reaction may be performed at a room temperature, such as about 20° C. or about 25° C.

Step 2 of Scheme 3 may be the same or similar as Step 1 of Scheme 1. Steps 1 and 2 of Scheme 3 may be performed simultaneously or in either sequential order.

Step 3 of Scheme 3 may involve reacting the compound of formula (3), which may be formed in Step 2 of Scheme 3, with the compound of formula (10), which may be formed in Step 1 of Scheme 3 to form a compound of formula (6):

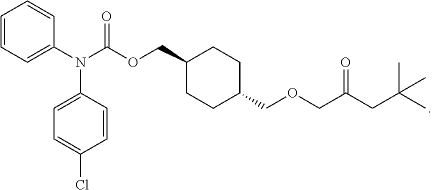 [SMILES: CC(C)(C)CC(=O)COC[C@H]1CC[C@H](COC(=O)N(c2ccccc2)c2ccc(Cl)cc2)CC1]

The compound of formula (3) may be used in such reaction without further crystalizing. The reaction may be performed in a solvent, such as an organic solvent, e.g. acetonitrile, tetrahydrofuran, 2-methyl tetrahydrofuran, toluene, or dichloromethane. A temperature for the reaction may be, for example, about 65° C.

Step 4 of Scheme 3 may involve converting the compound of formula (6) formed in Step 3 into a compound of formula (11):

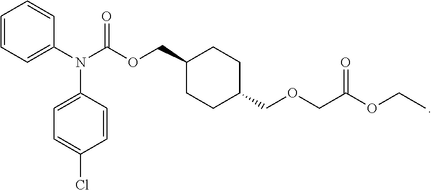 [SMILES: CCOC(=O)COC[C@H]1CC[C@H](COC(=O)N(c2ccccc2)c2ccc(Cl)cc2)CC1]

The compound of formula (6) formed in Step 3 may be used in such reaction with crystalizing. The reaction may be performed in the presence of an acidic catalyst, such as sulfuric acid (H₂SO₄). The reaction may be performed in a solvent, such as an organic solvent, e.g. an alcohol, such as methanol or ethanol or isopropanol. A temperature for the reaction may be, for example, about 73° C.

Step 5 of Scheme 3 may involve converting the compound of formula (11) into a product containing a salt compound of formula (7):

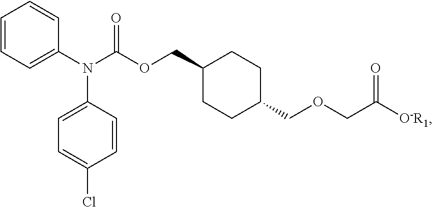

where R₁ may be a cation such as Na⁺, K⁺, or NH₄⁺. Such conversion may be performed by reacting the compound of formula (11) with an aqueous solution of a base, such as NaOH, KOH or NH₄OH. Such reaction may be performed at a room temperature, such as about 20° C. or about 25° C.

Step 6 of Scheme 3 may involve crystallizing the salt compound of formula (7) from the product of the reaction of Step 5 of Scheme 3 to improve the purity of the salt compound of formula 7 and/or to obtain the salt compound of formula 7 in a crystalline form. Such crystallization may be performed from an organic solvent, such as an aprotic organic solvent, e.g. acetone, alone or in mixture with water.

Step 7 of Scheme 3 may be the same or similar to Step 9 of Scheme 1 or Step 9 of Scheme 2.

Highlights and/or advantages of the process of Scheme 3 may be as follows:
(1) A convergent strategy was used to synthesize ralinepag (1) as shown in Scheme 3.
(2) The alkylation of diol (4) yielded mono t-butyl ester (10) in Step 1, which was reacted with activated aniline (3) to obtain ralinepag t-butyl ester (6) in Step 3.
(3) The use of mono t-butyl ester (10) in carbamate bond formation step (Step 3) instead of diol (4) alleviated the dimer impurity formation (1-3% formation in Scheme 1 vs <0.3% in Scheme 3 process) as only one hydroxy site (shown in red) is available for the reaction. This process avoided multiple crystallization steps (Steps 3 and 4) needed for isolation of pure carbamate (5) and dimer removal as given in Scheme 1 process. This resulted in reduction of the number of unit operations such as filtrations, drying and in-process testing. Hence, resulting in increased efficiency, safety, sustainability and yield in the process.
(4) The transesterification of hindered t-butyl ester to less hindered ethyl group using acid catalysis is the highlight of this reaction. Due to less hindered ethyl group, the need for harsh conditions was avoided and only 3.0 eq. of sodium hydroxide was used for the hydrolysis of ralinepag ethyl ester (9) improving the safety of the process. The carbamate impurity formation was also alleviated (7-8% in Scheme 1 process vs <0.6% in Scheme 3 process) in the hydrolysis step (Step 5) as relatively very low equivalents (42 eq. in Scheme 1 process vs 3 eq. in Scheme 3 process) of sodium hydroxide is sufficient. This reduced the burden on the subsequent acetone/water crystallization step (Step 6).
(5) No large excess of 50% aq. sodium hydroxide is used to hydrolyze and hence no concentrated HCl is needed for neutralization, increasing the process safety and scalability.
(6) Since low equivalents of sodium hydroxide and ethanol/water as solvent were used, the crude ralinepag sodium salt could be isolated from the reaction mixture in Step 5. The redundant sodium salt reformation step is avoided as the crude ralinepag sodium salt was directly isolated from the reaction mixture unlike Scheme 1 process.
(7) The number of steps in the process were reduced from 9 steps in Scheme 1 to 7 Steps in Scheme 3 process. The yield improved from ˜45% in Scheme 1 Process to ˜67% in Scheme 3 Process.

One or more of these advantages may be realized in the different embodiments depending on the specific reaction conditions and process steps.

In some embodiments, an alternative process of synthesizing ralinepag may involve:
(a) reacting a compound of formula (3):

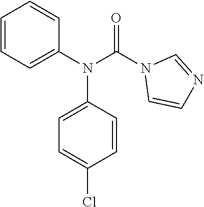 [SMILES: O=C(N(c1ccccc1)c1ccc(Cl)cc1)n1ccnc1]

with a compound of formula (10)

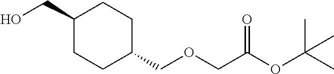 [SMILES: CC(C)(C)OC(=O)COC[C@H]1CC[C@H](CO)CC1]

to form a compound of formula (6):

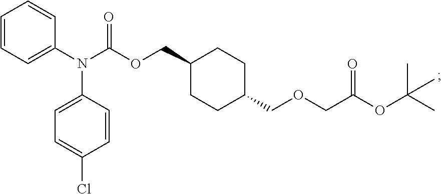 [SMILES: CC(C)(C)OC(=O)COC[C@H]1CC[C@H](COC(=O)N(c2ccccc2)c2ccc(Cl)cc2)CC1]

and
(b) reacting the compound of formula (6) with a base to form a salt compound of formula (7):

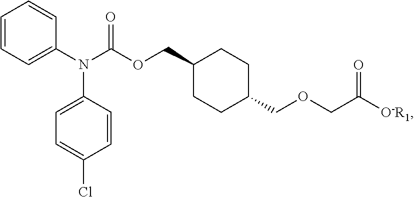

where R₁ is a cation, such as Na⁺, K⁺, and NH₄⁺.

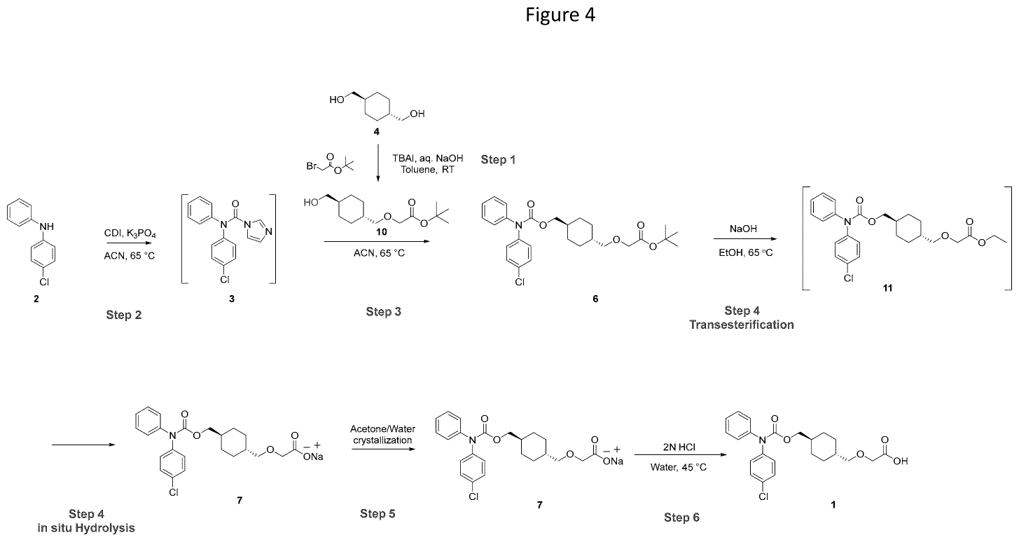

One embodiment of this alternative process is illustrated in Scheme 4 in FIG. 4.

Steps 1-3 of Scheme 4 may be the same or very similar as Steps 1-3 of Scheme 3.

Step 4 of Scheme 4 may involve reacting the compound of formula (6) formed in Step 3 to form a product containing ralinepag salt, i.e. the compound of formula (7). The base used in the reaction may be a diluted base (i.e. a base diluted in water), such as having 3.5 eq of base. The base may be, for example, diluted NaOH, diluted KOH or diluted NH₄OH. The reaction of Step 4 may involve initial transesterification of the compound of formula (6) resulting in a formation of an intermediate compound of formula (11), which hydrolyzes in situ into the salt compound of formula (7). The reaction of Step 4 may be performed in an organic solvent, such as ethanol, methanol, or isopropanol. A temperature for the reaction of Step 4 may be about 65° C.

Step 5 of Scheme 4 may involve crystallizing the product containing ralinepag salt, i.e. the compound of formula (7), formed in Step 4 to improve the purity of the salt compound of formula 7 and/or to obtain the salt compound of formula 7 in a crystalline form. Such crystallization may be performed from an organic solvent, such as an aprotic organic solvent, e.g., acetone, alone or in mixture with water.

Step 6 of Scheme 4 may be the same or similar to Step 7 of Scheme 3, Step 9 of Scheme 1 or Step 9 of Scheme 2.

Highlights and/or advantages of the process of Scheme 4 may be as follows:
(1) A convergent strategy was used to synthesize ralinepag (1) as shown in Scheme 4.
(2) The alkylation of diol (4) yielded mono t-butyl ester (10) in Step 1, which was reacted with activated aniline (3) to obtain ralinepag t-butyl ester (6) in Step 3. The ralinepag t-butyl ester was converted to isolable ralinepag sodium salt using a base (sodium hydroxide) in Step 4.
(3) The use of mono t-butyl ester (10) in carbamate bond formation step (Step 3) instead of diol (4) alleviated the dimer impurity formation (1-3% formation in Scheme 1 vs <0.3% in Scheme 4 process) as only one hydroxy site (shown in red) is available for the reaction. This process avoided multiple crystallization steps (Steps 3 and 4) needed for isolation of pure carbamate (5) and dimer removal as given in Scheme 1 process. This resulted in reduction of the number of unit operations such as filtrations, drying and in-process testing.
(4) One of highlights of this process may be base catalyzed transesterification of hindered t-butyl ester to less hindered ethyl ester, which is easier to hydrolyze under mild (about 3.5 eq. of base) conditions. The formation ralinepag ethyl ester during the reaction was observed by TLC and HPLC analysis. In case of Scheme 1 Process, harsh (about 42 eq. of base) condition is needed to hydrolyze the hindered t-butyl ester which leads to the formation of carbamate impurity in about 7-8% range. This reduction in carbamate impurity formation decreased the burden on the subsequent acetone/water crystallization step (Step 5).
(5) The harsh conditions were avoided as only about 3.5 eq. of sodium hydroxide was used for the hydrolysis of ralinepag ethyl ester (9) improving the safety of the process. No large excess of 50% aq. sodium hydroxide is used to hydrolyze and hence no conc. HCl is needed for neutralization.
(6) Since low equivalents of base and non-aqueous conditions were used, the crude ralinepag sodium salt could be isolated from the reaction mixture in Step 4. The redundant sodium salt reformation step is avoided as the crude ralinepag sodium salt was directly isolated from the reaction mixture unlike Scheme 1 process.
(7) The number of steps in the process were reduced from 9 steps in Scheme 1 to 6 Steps in Scheme 4 process. The yield improved from ˜45% in Scheme 1 Process to ˜79% in Scheme 4 Process. The reduced number of steps in this process decreased the number of unit operations needed in the process, such as filtrations, drying and in-process testing. Hence, resulting in increased efficiency, safety, sustainability and yield in the process.

One or more of these advantages may be realized in the different embodiments depending on the specific reaction conditions and process steps.

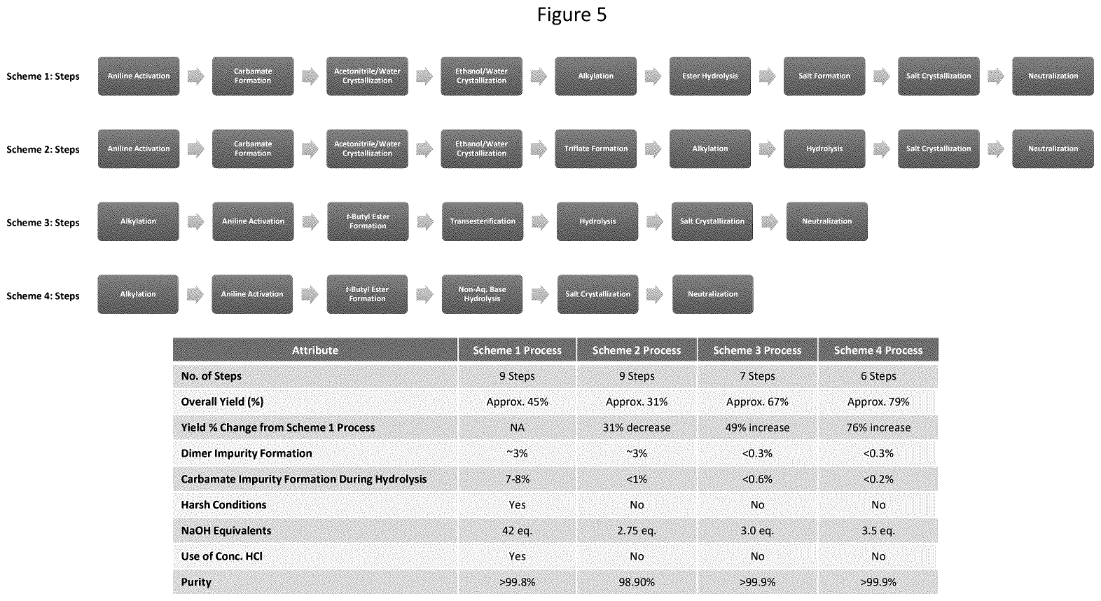

FIG. 5 provides comparison between processes of Schemes 1-4.

The present disclosure also provides high purity batches of ralinepag as well as high purity batches of intermediates used in Schemes 2-4. The batches may have a size of at least 50 g, at least 100 g, at least 200 g, at least 300 g, at least 400 g, at least 500 g, at least 600 g, at least 700 g, at least 800 g, at least 900 g or at least 1 kg, at least 5 kg or at least 10 kg. The batches may have a purity of at least 98%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9% or more than 99.9%

In one embodiments, a batch of a compound of formula (6):

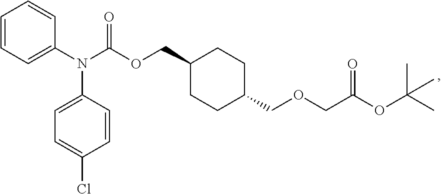 [SMILES: CC(C)(C)OC(=O)COC[C@H]1CC[C@H](COC(=O)N(c2ccccc2)c2ccc(Cl)cc2)CC1]

may contain less than 1% or less than 0.9% or less than 0.8% or less than 0.7% or less than 0.6% or less than 0.5% or less than 0.4% or less than 0.3% or less than 0.2% or less than 0.1% of a dimer impurity of the following formula:

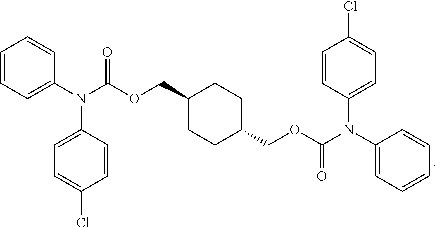 [SMILES: O=C(OC[C@H]1CC[C@H](COC(=O)N(c2ccccc2)c2ccc(Cl)cc2)CC1)N(c1ccccc1)c1ccc(Cl)cc1]

The batch may have a size of at least 50 g, at least 100 g, at least 200 g, at least 300 g, at least 400 g, at least 500 g, at least 600 g, at least 700 g, at least 800 g, at least 900 g or at least 1 kg, at least 5 kg or at least 10 kg.

In another embodiment, a batch of a compound of formula (1):

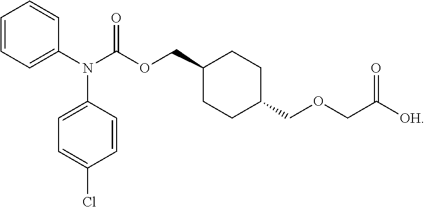 [SMILES: O=C(O)COC[C@H]1CC[C@H](COC(=O)N(c2ccccc2)c2ccc(Cl)cc2)CC1]

may have a purity of at least 99.9%. The batch may have a size of at least 50 g, at least 100 g, at least 200 g, at least 300 g, at least 400 g, at least 500 g, at least 600 g, at least 700 g, at least 800 g, at least 900 g or at least 1 kg, at least 5 kg, or at least 10 kg. In some embodiments, the batch of the compound of formula (1) may contain below 0.01% of aniline (2) impurity. In some embodiments, the batch of the compound of formula (1) may contain below 0.01% of carbamate (5) impurity. In some embodiments, the batch of the compound of formula (1) may contain below 0.01% of each of aniline (2) impurity and carbamate (5) impurity.

The present disclosure also provides a method of high purity ralinepag from a crude ralinepag salt. The method may involve (a) neutralizing the crude ralinepag salt to form crude ralinepag and (b) crystallizing crude ralinepag to form high purity ralinepag, which may have a purity of at least 99%.

As used herein, the term “crude” refers to a crude product of a reaction directly after the reaction is complete without any purification of the desired. The crude product is a mixture containing the desired product along with any impurities, byproducts, and/or excess reactants.

The crude ralinepag salt formed in Step 7 of Scheme 1, Step 7 of Scheme 2, Step 5 of Scheme 3 or Step 4 of Scheme 4. Thus, neutralizing (a) and crystallizing (b) may replace the last two steps in each of Schemes 1-4. Such replacement may lead to an improved impurity profile in the produced high purity ralinepag. The produced high purity ralinepag may have a reduced content of impurities. For example, for Scheme 4 modified by replacing the last two steps with neutralizing (a) and crystallizing (b) the produced high purity ralinepag may have a reduced content of impurities, such as aniline (2) impurity and carbamate (5) impurity, below 0.05%.

Neutralizing (a) may be performed by reacting the crude ralinepag salt with an acid, such as HCl. For example, neutralizing (a) may be performed by reacting the crude ralinepag salt with 2N HCl.

Crystallizing (b) may be performed using a solvent, which may be a mixture of an aprotic solvent, such as acetone, with water. For example, the solvent may be a mixture of 60% acetone and water.

Embodiments described herein are further illustrated by, though in no way limited to, the following working examples.

### Examples

### Synthesis of Ralinepag from Carbamate Triflate (Scheme 2)

### Synthesis of Carbamate Triflate (8)

Step 5: To a solution of carbamate (5) (20.00 g, 53.49 mmol) in dichloromethane (214 mL) was added 2,6-lutidine (8.68 mL, 74.89 mmol) at RT under argon. To this mixture at −20° C., trifluoromethanesulfonic anhydride (10.78 mL, 64.19 mmol) was added dropwise using a dropping funnel. The reaction mixture turned pink from dark orange. The reaction mixture was stirred at −20° C. for 2 h and TLC. The reaction mixture was warmed to 0° C. and was quenched with water (100 mL). The organic layer was separated and washed with water (100 mL), 1N HCl (200 mL), water (200 mL), brine (200 mL), dried on sodium sulfate (20 g) and evaporated in vacuo to obtain crude carbamate triflate (8) (25.85 g, 95.52% yield). The crude product was characterized by spectral data IR, ¹H NMR, ¹³CNMR, ¹⁹F NMR.

### Synthesis of Ralinepag Sodium Salt (7)

Step 6: To a solution of methyl glycolate (0.43 mL, 5.53 mmol) in tetrahydrofuran (10 mL) at −20° C. was added sodium bis(trimethylsilyl)amide solution in 1.0 M THF (6.3 mL, 6.32 mmol) dropwise over 10 min and stirred for another 10 min under argon. To this mixture at−20° C. was added a solution of carbamate triflate (8) (2.0 g, 3.95 mmol) in tetrahydrofuran (12 mL). This was allowed to warm to RT and stirred for 3 h. The reaction was found to be complete by TLC.

Step 7: To the above reaction mixture was added a solution of sodium hydroxide (0.43 g, 10.86 mmol) in water (7 mL) and stirred for 1.5 h. The reaction was found to be complete by TLC. At this stage, the reaction was evaporated in vacuo to remove organic volatiles and to reduce the water content (5 mL). To this isopropanol (15 mL) and acetone (17 mL) was added and ralinepag sodium salt precipitated out. The pH was found to be 13.59 and this was adjusted to 9.36 by adding 2N HCl (2.5 mL). This was cooled to 0° C., stirred for 15 min and the precipitated solid was filtered using a Buchner funnel and the solids were washed with acetone (20 mL) to obtain dry crude ralinepag sodium salt (7) (1.29 g) with a purity of 96.4% by HPLC.

Step 8: The above crude ralinepag sodium salt (7) (1.29 g) was crystallized from acetone (15.6 mL) and water (4.6 mL) at 65° C. and cooled to 0° C. to obtain pure ralinepag sodium salt (7) (1.16 g) with 98.85% purity by HPLC.

### Synthesis of Ralinepag (1)

Step 9: To a suspension of ralinepag sodium salt (7) (1.0 g, 2.20 mmol) in water (13 mL) was added 2N hydrochloric acid (1.3 mL, 2.60 mmol) and heated to 48° C. (bath temp) for 2.5 h. The solids were filtered through Buchner funnel and washed the solids with water (35 mL) until pH 6.7 to obtain ralinepag (1) (0.74 g) with 98.90% HPLC purity. This product was characterized by ¹HNMR, ¹³C NMR, LC-MS, and IR. The melting point was found to be 127.5° C. to 128.8° C.

### Synthesis of Mono t-Butyl Ester (10): a Common Starting Material for Process of Scheme 3 and 4

Step 1: A 30-L reactor was charged with trans-1,4-cyclohexanedimethanol (4) (1000 g, 6.93 mol), TBAI (640.3 g, 1.73 mol) and toluene (10 L), followed by addition of aq. sodium hydroxide (ION, 1390 mL, 13.90 mol). The mixture was stirred for 10 min at RT and then tert-butyl bromoacetate (1495.5 g, 7.67 mol) was added and the mixture was stirred at room temperature for 8 h. The reaction progress was monitored by TLC. The organic layer was separated and washed with water (2×2.5 L). The combined aqueous layers were extracted with MTBE (2×3 L) and this MTBE layer was washed with water (1×2 L) and brine (1×2 L), concentrated in vacuo to give 1.54 kg of crude product. The crude product was purified on silica gel column to obtain pure mono t-butyl ester (10) (856.6 g, yield 47.7%) with a GC purity greater than 99%. The product was characterized by IR, ¹H NMR, ¹³C NMR & GC.

### Experimental for Synthesis of Ralinepag Via Acid Catalyzed Transesterification (Scheme 3)

### Synthesis of Ralinepag tert-Butyl Ester (6)

Step 2 and 3: To a 5-L reactor was added 4-chloro-N-phenylaniline (2) (100.0 g, 491.0 mmol) and anhydrous acetonitrile (600 mL). To this solution was added anhydrous potassium phosphate (31.27 g, 147.31 mmol) and acetonitrile (200 mL) followed by N,N-carbonyldiimidazole (CDI) (87.58 g, 540.12 mmol) and acetonitrile (200 mL) at room temperature under argon. The reaction mixture was heated to 65° C. for 4 h and checked TLC for completion. To this intermediate was added a solution of mono t-butyl ester (10) (126.85 g, 491.0 mmol) in anhydrous acetonitrile (200 mL) under argon. After complete addition, the reaction mixture was continued to stir and heat at 65° C. under argon. After 16 h, the reaction mixture was checked by TLC and was found to be complete. The mixture was filtered through a pad of Celite (38 g) and washed the solid with acetonitrile (2×250 mL). The filtrate was concentrated in vacuo to give crude product (313.29 g). The crude product (310.29 g) was dissolved in toluene (1000 mL) and washed with 2N hydrochloric acid (500 mL), water (2×500 mL) and brine (500 mL). The organic layer was separated and evaporated in vacuo to give crude ralinepag tert-butyl ester (6) (253.44 g). The crude product was characterized by ¹H NMR.

### Synthesis of Ralinepag Ethyl Ester (6)

Step 4: The ralinepag tert-butyl ester (6) from Step 3 was transferred into a 5-L reactor, then a solution of sulfuric acid, 95-98% (5 mL) in ethyl alcohol (100 mL) was added at 19.1° C. This was heated to 72.6° C. and the reaction was monitored by TLC. The reaction was complete and cooled to 20° C. This crude ralinepag ethyl ester (11) in ethanolic solution was used in the next step of base hydrolysis.

### Synthesis of Ralinepag Sodium Salt (7)

Step 5: To the above solution of ralinepag tert-butyl ester (6) in ethyl alcohol was added a solution of sodium hydroxide (58.9 g, 1472.5 mmol) in water (250 mL) slowly. After complete addition, the reaction mixture was stirred at room temperature and monitored the reaction by TLC. The solid product was filtered through a Buchner funnel and washed with ethyl alcohol (4×250 mL) and dried to obtain crude ralinepag sodium salt (7) (175 g) with a HPLC purity of 98.5%.

### Crystallization of Ralinepag Sodium Salt (7)

Step 6: To a 5-L reactor was added acetone (1934 mL) and water (567 mL) followed by crude ralinepag sodium salt (7) (173.8 g). The mixture was stirred and heated to 58° C. for 1 h and the mixture was not clear, then water (200 mL) was added at this temperature until a clear solution was obtained. The solution was filtered, acetone (1898 mL) and water (75 mL) were added and heated to 64° C. to a clear solution. This was cooled to cloud point and stirred for 1 h at cloud point temperature. This was then cooled to 0° C. and stirred overnight. The solid ralinepag sodium salt was collected in a Buchner funnel and washed with acetone (1200 mL). The product was dried to obtain pure ralinepag sodium salt (7) (150 g) with a HPLC purity of 100%.

### Synthesis of Ralinepag (1)

Step 7: To a 5-L reactor was added water (1945 mL) followed by ralinepag sodium salt (7) (148.9 g) and the white suspension was heated to 25° C. To this 2N hydrochloric acid (208 mL) was added and heated to 45° C. for 2 h. The solids were cooled to RT, filtered through a Buchner funnel, and washed with water (2500 mL) until the filtrate was neutral. The wet cake was dried in oven under vacuum to obtain ralinepag (1) (139 g) with a HPLC purity of 100%. The melting point was found to be 129-130° C.

### Experimental for Synthesis of Ralinepag (1) Via Non-Aqueous Base Hydrolysis (Scheme 4)

### Synthesis of Ralinepag tert-butyl ester (6)

Step 2: To a 15-L reactor was added 4-chloro-N-phenylaniline (2) (500.0 g, 2.46 mol) and anhydrous acetonitrile (1000 mL) under argon. To this clear solution was added anhydrous potassium phosphate (157.0 g, 0.74 mol) and acetonitrile (500 mL) followed by N,N-carbonyldiimidazole (CDI) (418.0 g, 2.58 mol) and acetonitrile (1000 mL). The reaction mixture was heated to 65.2° C. for 3 h 6 min and checked by TLC for completion. To this intermediate was added a solution of mono t-butyl ester (10) (667.0 g, 2.53 mol) in anhydrous acetonitrile (3500 mL). After complete addition, the reaction mixture was continued to stir at 65.2° C. After 17 h 20 min, the reaction mixture was checked by TLC and found to be complete. The reaction mixture was cooled to room temperature, filtered through a pad of Celite (99.6 g) and washed the solid with acetonitrile (1000 mL). The filtrate was evaporated in vacuo and the residue was chased with MTBE (1000 mL). The crude product was dissolved in MTBE (5000 mL), washed with 2N hydrochloric acid (5000 mL) water (2×5000 mL). The organic layer was separated, evaporated in vacuo to give crude ralinepag tert-butyl ester (6) (1150 g). This product was used for the next step as such.

### Synthesis of Ralinepag Sodium Salt (7)

Step 4: To crude ralinepag tert-butyl ester (6) (1149 g) in ethyl alcohol (7500 mL) at 45° C. was added a solution of sodium hydroxide (344 g) in ethyl alcohol (5000 mL). The reaction mixture was heated to 65.4° C., stirred for 1 h and TLC indicated completion of the reaction. The reaction mixture was cooled to RT and the solids were filtered and washed with ethyl alcohol (5000 mL) followed by acetone (10000 mL). The product was dried under in vacuum oven at 64° C. to obtain crude ralinepag sodium salt (7) (1018 g) with a HPLC purity of 99.01%.

### Crystallization of Ralinepag Sodium Salt

Step 5: To a clean 15-L jacketed reactor was added acetone (11274 mL) and process water (4444 mL) followed by crude ralinepag sodium salt (7) (1013.0 g). The mixture was heated to 57.1° C. for 1 h 5 min and the mixture was clear. The hot solution was filtered through a depth filter and acetone (9052 mL) was added. The mixture was cooled to cloudy point and stirred at cloud point temperature for 1 h. This cooled slowly to 0° C. and stirred for overnight. After 16 h, the crystals were filtered, washed with acetone (8000 mL) and dried in vacuum oven at 65° C. to obtain pure ralinepag sodium salt (7) (888.2 g) with a HPLC purity of 100%.

### Neutralization of Ralinepag Sodium Salt (7) to Ralinepag (1)

Step 6: To a clean 15-L reactor was added ralinepag sodium salt (7) (870 g) and water (11310 mL). The white suspension was stirred and heated to reached 22° C., 2N hydrochloric acid (1217 mL) was added and the mixture was heated to 45.1° C. for 2 h. The solids were filtered, washed with water (15000 mL) and dried in the oven at 65° C. to obtain pure ralinepag (1) (812 g) with a HPLC purity of 100%. The pure ralinepag was characterized by spectral data IR, ¹H NMR, ¹³C NMR and LCMS. The melting point was found to be 129-130° C.

### Modified Process

In the modified process, steps 5 and 6 were replaced with the following modified steps 5 and 6:

### Neutralization of Crude Ralinepag Sodium Salt (7) to Crude Ralinepag (1)

Modified Step 5: To a clean 100-mL RB flask was added crude ralinepag sodium salt (7) (5.0 g, 11.02 mmol) and water (WFI) (60 mL, 12 vol based on 7) at room temperature. The pasty mixture was stirred at 59.1° C. It was cooled to 50.2° C. and then 2N hydrochloric acid (7.0 mL, 14.0 mmol) was added. The mixture was stirred and heated to 53° C. for 4 h. It was cooled to room temperature, filtered and washed the solid with water (7×50 mL). The solid was dried in vacuum oven at 64.4° C./4 mbar for 4 h 50 min to obtain crude ralinepag (1) (4.42 g). The purity of crude ralinepag (1) by HPLC was 99.25% (ralinepag) along with impurities 0.10% (dimer), 4-chloro-N-phenylaniline (0.39%), carbamate (5) (0.17%) and unknown (0.09%).

### Crystallization of Crude Ralinepag (1) to Pure Ralinepag (1)

Modified Step 6: To a clean 100-mL RB flask was added crude ralinepag (1) (4.0 g, 9.26 mmol), pre-mixed solution of acetone (36 mL, 9 vol) and water (WFI) (24 mL, 6 vol). The suspension was heated slowly to 61.9° C. for 30 min to obtain a clear solution. The solution was cooled slowly to room temperature and stirred at RT overnight. The resulting crystals were filtered, washed with water (3×25 mL), dried in vacuum oven at 64.4° C./4 mbar for 2 h to obtain pure ralinepag (1) (3.89 g) with melting point: 129-130° C. The purity of pure ralinepag (1) by HPLC was 99.90% with 0.10% dimer impurity.

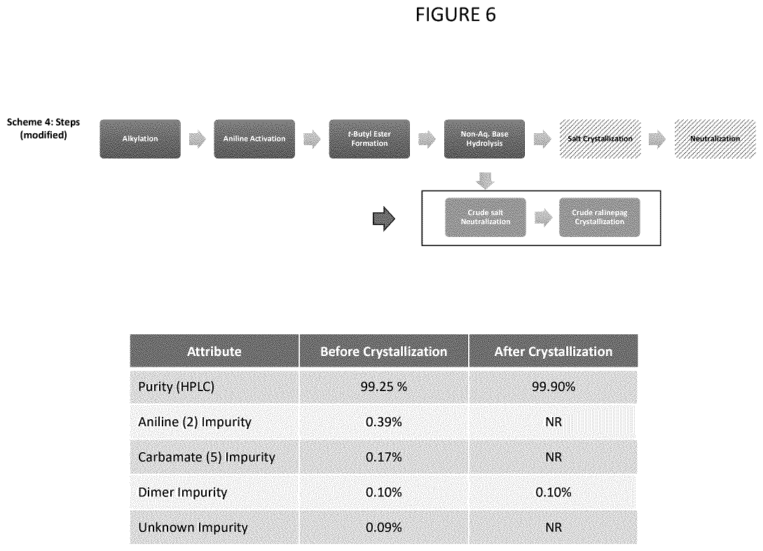

The table in FIG. 6 provides a comparison between impurity content (a) after modified step 5 but before modified step 6 and (b) after modified step 6. Crystallization of modified step 6 reduced and/or removed each of aniline (2) impurity, carbamate (5) impurity and unknown impurity to levels beyond reportable limit (0.05%).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of synthesizing a compound of formula (1): 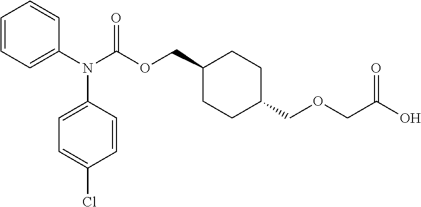 [SMILES: O=C(O)COC[C@H]1CC[C@H](COC(=O)N(c2ccccc2)c2ccc(Cl)cc2)CC1]
or a salt thereof, the method comprising:
(a) converting a carbamate compound of formula (5): 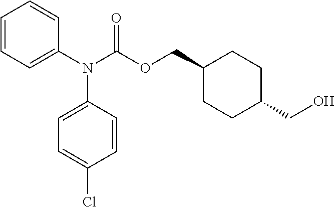 [SMILES: O=C(OC[C@H]1CC[C@H](CO)CC1)N(c1ccccc1)c1ccc(Cl)cc1]
into a triflate compound of formula (8): 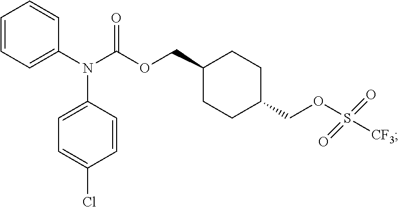 [SMILES: O=C(OC[C@H]1CC[C@H](COS(=O)(=O)C(F)(F)F)CC1)N(c1ccccc1)c1ccc(Cl)cc1]
(b) converting the triflate compound of formula (8) into a methyl ester compound of formula (9): 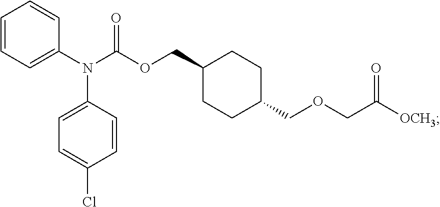 [SMILES: COC(=O)COC[C@H]1CC[C@H](COC(=O)N(c2ccccc2)c2ccc(Cl)cc2)CC1]
and
(c) converting the methyl ester compound of formula (9) into a salt compound of formula (7): 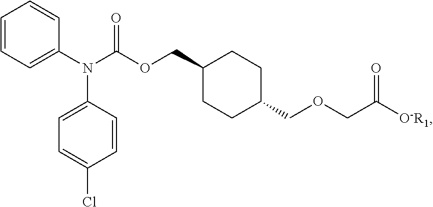
wherein R₁ is a cation selected from Na⁺, K⁺, and NH₄⁺.

2-10. (canceled)

11. A method of synthesizing a compound of formula (1): 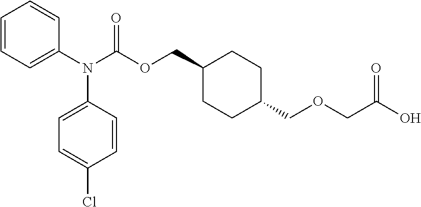 [SMILES: O=C(O)COC[C@H]1CC[C@H](COC(=O)N(c2ccccc2)c2ccc(Cl)cc2)CC1]
or a salt thereof, the method comprising:
(a) reacting a compound of formula (3): 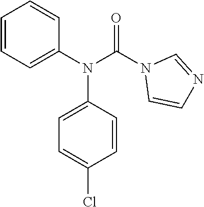 [SMILES: O=C(N(c1ccccc1)c1ccc(Cl)cc1)n1ccnc1]
with a compound of formula (10) 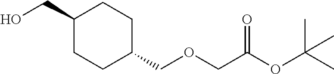 [SMILES: CC(C)(C)OC(=O)COC[C@H]1CC[C@H](CO)CC1]
to form a compound of formula (6): 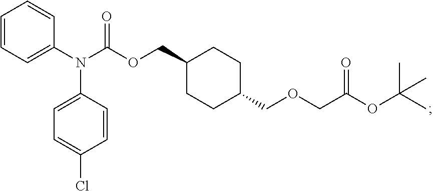 [SMILES: CC(C)(C)OC(=O)COC[C@H]1CC[C@H](COC(=O)N(c2ccccc2)c2ccc(Cl)cc2)CC1]
(b) converting the compound of formula (6) into a compound of formula (11): 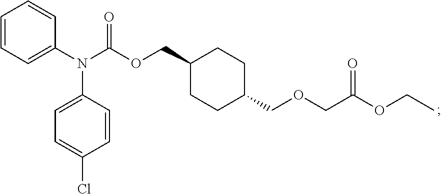 [SMILES: CCOC(=O)COC[C@H]1CC[C@H](COC(=O)N(c2ccccc2)c2ccc(Cl)cc2)CC1]
and
(c) converting the compound of formula (11) into a salt compound of formula (7): 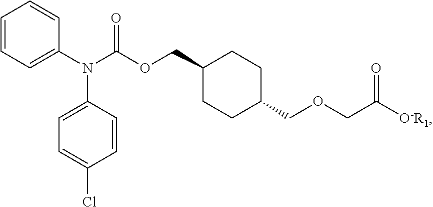
wherein R₁ is a cation selected from Na⁺, K⁺, and NH₄⁺.

12. The method of claim 11, wherein said converting (b) is performed in a presence of an acid.

13. The method of claim 12, which the acid is a sulfuric acid.

14. The method of claim 11, wherein said converting (c) comprises hydrolyzing the compound of formula (11) to form the salt compound of formula (7).

15. The method of claim 14, further said converting (11) further comprises crystallizing a product of said hydrolyzing.

16. The method of claim 15, wherein said crystallizing is performed in a solvent comprising acetone and water.

17. The method of claim 14, further comprising neutralizing a product of said hydrolyzing with an acid.

18. The method of claim 17, further comprising crystalizing the compound of formula (1) from a product of said neutralizing.

19. The method of claim 18, wherein said crystallizing is performed in a solvent comprising acetone and water.

20. A method of synthesizing a compound of formula (1): 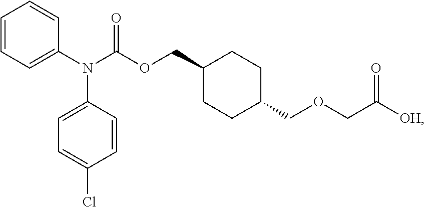 [SMILES: O=C(O)COC[C@H]1CC[C@H](COC(=O)N(c2ccccc2)c2ccc(Cl)cc2)CC1]
the method comprises:
(a) reacting a compound of formula (3): 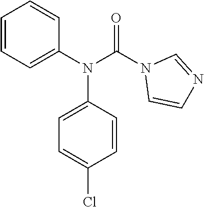 [SMILES: O=C(N(c1ccccc1)c1ccc(Cl)cc1)n1ccnc1]
with a compound of formula (10) 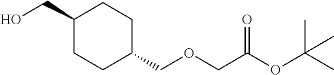 [SMILES: CC(C)(C)OC(=O)COC[C@H]1CC[C@H](CO)CC1]
to form a compound of formula (6): 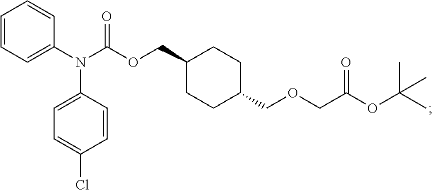 [SMILES: CC(C)(C)OC(=O)COC[C@H]1CC[C@H](COC(=O)N(c2ccccc2)c2ccc(Cl)cc2)CC1]
and
(b) reacting the compound of formula (6) with a base to form a salt compound of formula (7): 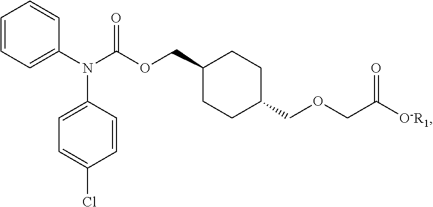
wherein R₁ is a cation selected from Na⁺, K⁺, and NH₄⁺.

21-25. (canceled)

26. The method of claim 11, further comprising reacting a compound of formula (4): 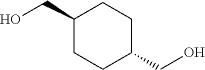
with tert-butyl bromoacetate to form the compound of formula (10).

27. The method of claim 11, further comprising converting the salt compound of formula (7) into the compound of formula (1).

28. A compound of formula (10):

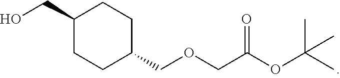 [SMILES: CC(C)(C)OC(=O)COC[C@H]1CC[C@H](CO)CC1]

29. A compound of formula (11):

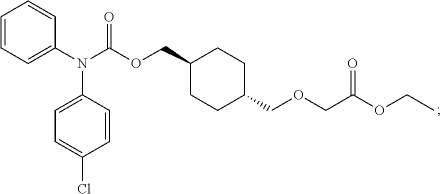 [SMILES: CCOC(=O)COC[C@H]1CC[C@H](COC(=O)N(c2ccccc2)c2ccc(Cl)cc2)CC1]

30. A batch of a compound of formula (6): 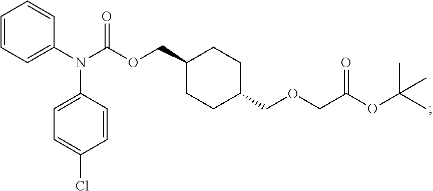 [SMILES: CC(C)(C)OC(=O)COC[C@H]1CC[C@H](COC(=O)N(c2ccccc2)c2ccc(Cl)cc2)CC1]
wherein said batch contains less than 1% of a dimer impurity of the following formula:

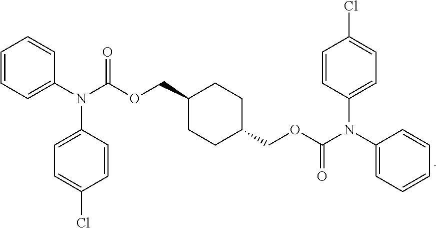 [SMILES: O=C(OC[C@H]1CC[C@H](COC(=O)N(c2ccccc2)c2ccc(Cl)cc2)CC1)N(c1ccccc1)c1ccc(Cl)cc1]

31. A batch of a compound of formula (1): 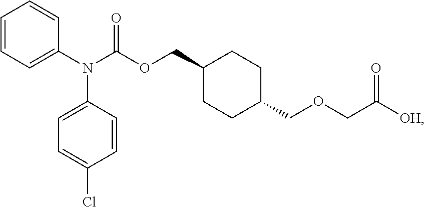 [SMILES: O=C(O)COC[C@H]1CC[C@H](COC(=O)N(c2ccccc2)c2ccc(Cl)cc2)CC1]
wherein said batch has purity of at least 99.9%.

32. A method of producing a compound of formula (1): 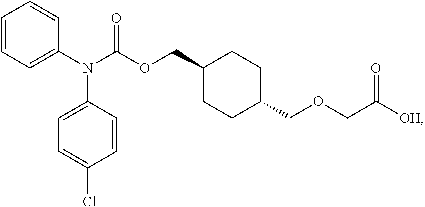 [SMILES: O=C(O)COC[C@H]1CC[C@H](COC(=O)N(c2ccccc2)c2ccc(Cl)cc2)CC1]
the method comprising:
(a) reacting a crude product comprising a salt compound of formula (7): 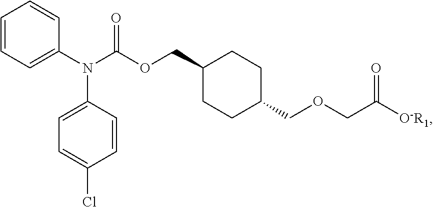
wherein R₁ is a cation selected from Na⁺, K⁺, and NH₄⁺ with an acid to form a crude product comprising the compound of formula (1); and
(b) crystallizing the compound of formula (1) from the crude product formed in the reacting.

33-34. (canceled)

35. The method of claim 12, wherein said converting (c) comprises hydrolyzing the compound of formula (11) to form the salt compound of formula (7).

36. The method of claim 13, wherein said converting (c) comprises hydrolyzing the compound of formula (11) to form the salt compound of formula (7).
